(19) United States
(12) Patent Application Publication
(10) Pub. No.: US 2010/0249753 A1
(43) Pub. Date: Sep. 30, 2010

(54) Vented syringe system and method for the containment, mixing and ejection of wetted particulate material

(76) Inventors: David M. Gaisser, Gainesville, FL (US); Srinivas Katta, Alachua, FL (US)

Correspondence Address: ROGERS TOWERS, P.A., 1301 RIVERPLACE BOULEVARD, SUITE 1500, JACKSONVILLE, FL, 32207, US

(21) Appl. No.: 12/660,395

(22) Filed: Feb. 25, 2010

Related U.S. Application Data
(60) Provisional application No. 61/208,561, filed on Feb. 25, 2009.

(51) Int. Cl. A61M 5/32

(52) U.S. Cl. 604/519; 604/85

(57) ABSTRACT

A vented syringe system and method for particulate containment, mixing and delivery, the system having a vented syringe containing particulate material structured to enable entrapped gases to escape during the injection of liquid into the particulate material, such that gas is removed from the vented syringe prior to ejection of the wetted particulate material.

This application claims the benefit of U.S. Provisional Patent Application Ser. No. 61/208,561, filed Feb. 25, 2009.

### BACKGROUND OF THE INVENTION

The present invention relates generally to the field of syringe-type medical device instrumentation and methods, and more specifically to a medical device containment, mixing and ejection system for the preparation and delivery of wetted particulate materials, and its method of use. Such a system may be used, for example, for the preparation and application of particulate synthetic bone graft devices.

In medicine, there is often a need for the application of a graft material to a bone defect that may have originated from disease, surgery, or traumatic injury. Such bone graft materials may consist of either organic tissue or synthetic materials. Organic tissue may include human bone tissue collected either from the patient or from a donor, either human (i.e. cadaveric) or animal sources.

Synthetic materials, those not involving human and/or animal tissues, can be of several different forms. Some materials, such as metals and certain ceramics, are intended as bulk devices for permanent replacements to the original bone, supporting the applied loads and bone function. These, however, change the physiologic balance at the graft site and may impair or impeded healing of the graft site. Other materials, such as calcium sulfate ceramics, may be implanted as slurries or cements. These materials fill the graft site and harden, preventing repair of the defect due to their physical presence. While some of these materials are permanent, others are temporary and are absorbed. If this absorption is too rapid, such as with calcium sulfate ceramics, the material will be absorbed before new bone tissue can form and heal.

A third forth for synthetic graft materials consists of particulate devices. These devices, typically polymeric, glass, or ceramic in nature, consist of aggregations of discrete particles. The spaces between the particles permit defect healing by virtue of new bone tissue formation and ingrowth. One such particulate material, bioactive glass, has been demonstrated to directly stimulate the cells necessary for bone formation when in particulate form. As a preferred example, one specific bioactive glass composition, termed Bioglass®, has a composition of 21% silicon, 18% calcium, 18% sodium, 3% phosphorus, and 40% oxygen (by weight percent). This Bioglass material has been used clinically for over 15 years as a particulate bone graft device.

One of the drawbacks with particulate bone graft devices is their preparation. Most particulate materials are supplied in a cup or bottle, from which they are extracted, placed in a sterile basin or bowl, and mixed with an activating or wetting agent. Extensive manual manipulation of the material may be required to wet the entire device properly. Similarly, variations in device wetting may occur due to differing amounts of fluid being added with each device preparation. Another drawback with this method of mixing is that after mixing, the wetted material must be manually removed from the basin and placed in the graft site with a surgical instrument such as a rongeur or spatula. Frequently, this transfer can result in partial loss of graft material volume due either to the inability to extract all the material from the mixing basin or to actual dropping of the material during handling.

One method to modify the handling and preparation of particulate materials is via their packaging and delivery vehicle. Some particulate devices, after dispensing and mixing in the sterile basin with the appropriate wetting agent, can be loaded into a syringe and expressed into the desired graft site. However, the preparation of these materials is still performed in a basin, requiring additional handling to reload the material into the device syringe.

To address the material handling issues, some devices are packaged in a syringe delivery system. One bioactive glass particulate device is available in a syringe format, the syringe having a porous screen cap over the end. To prepare the device, the end of the syringe with the screen cap is placed in the desired wetting fluid. On pulling back the syringe plunger, the vacuum created draws fluid up through the cap and into the graft material. Such systems are taught by multiple patents, including U.S. Pat. Nos. 5,181,918 and 6,554,803. One drawback to such systems is that the entire volume of the syringe cannot be used to contain the desired particulate material; the length of the syringe barrel must be at least twice that of the volume of graft material contained, otherwise the syringe will not be able to draw up sufficient fluid to completely wet the device. Once the material is wetted, this excess air must either be removed or forced through the wetted graft material on expression. In the case of U.S. Pat. No. 5,181,918, a separate valve and piston arrangement is included to remove this entrapped air from behind the wetted material.

Another particulate bone graft device packaged in a syringe more completely wets the device material, but requires a cumbersome array of equipment to accomplish. Instead of filling the syringe with fluid from the open end, this device is wetted via attachment of a second fluid syringe to the plunger seal at the base of the device particulate syringe. The device is wetted by expressing the wetting fluid into the particulate through the plunger seal. The fluid syringe is removed and a second plunger shaft is attached to the syringe seal. The device is then expressed by pressing on the plunger shaft. Such a syringe device is, in part, taught by U.S. Pat. No. 4,551,135, which presents a syringe having a means to attach a second syringe to the plunger to aid in fluid administration and mixing.

Other syringe devices have been designed for use with particulate materials, but none fully address all the issues. U.S. Pat. No. 4,405,249 shows a vented mixing syringe with apertures in the piston to allow for gas escape. U.S. Pat. No. 6,592,251, shows a mixing apparatus that utilizes two syringes.

To address issues raised by prior devices, a vented particulate mixing syringe device according to the present invention substantially departs from the conventional concepts and designs of the prior art, and in so doing, provides a device developed primarily for use as a mixing system for particulate bone graft materials, resulting in improved device preparation and delivery for the healing of bone defects.

### SUMMARY OF THE INVENTION

In view of the foregoing disadvantages inherent with the known methods of bone graft device preparation and delivery present in the prior art, the present invention provides a new containment, mixing and delivery system for bone graft materials that improves device handling and can be readily applied to particulate bone graft materials to enhance material bone defect healing.

The general purpose of the present invention, which will be described subsequently in greater detail, is to provide a new containment mixing and delivery method and system for particulate bone graft materials that has many advantages over methods mentioned heretofore and many novel features that result in a new bone graft delivery device which is not anticipated, rendered obvious, suggested, or implied by any of the prior are bone graft materials, either alone or in any combination thereof.

To attain this, the present invention generally comprises an open-bore syringe, having a plunger, a barrel, a nozzle member mounted onto the distal open end of the barrel, a cap member mounted on nozzle to retain the device particulate, and a series of vents in the barrel of the syringe to permit release of entrapped air during instilling of a device wetting fluid. The wetting fluid is administered into the device through the nozzle member after removal of the cap member. The plunger is retracted or moved in the proximal direction during fluid instillation such that the vents are exposed, resulting in release of any entrapped air. The device is fully wetted when fluid begins to escape through the vent holes. Once the material has been wetted, the nozzle member is removed and the wetted particulate material expressed by depressing the plunger in the distal direction.

In this respect, before explaining at least one embodiment of the invention in detail, it is understood that the invention is not limited in its application to the details of application or the proportions thereof as set forth in the following description. The invention is capable of other similar embodiments. Also, it is to be understood that the phrasing and terminology employed herein are for the purpose of the description and should not be regarded as limiting.

The primary object of the present invention is to provide a device for the wetting of particulate materials and their subsequent delivery that will overcome the shortcomings of prior art devices.

An object of the present invention is to provide a syringe device for the wetting and preparation of particulate bone graft materials that will permit wetting of the material throughout the full volume of the graft material.

Another object of the invention is to provide a syringe device for the delivery of a prepared particulate bone graft material to the desired osseous graft site without further manual manipulation or handling of the prepared material.

Other objects and advantages of the present invention will become obvious to the reader and it is intended that these objects and advantages are within the scope of the present invention.

### BRIEF DESCRIPTION OF THE DRAWINGS

FIG. 1 is an expanded view of an embodiment of the vented syringe.

FIG. 2 is a view of the assembled embodiment of FIG. 1 in the pre-wetted configuration with the plunger seal disposed to the distal side of the vent holes, shown partially in cross-section to expose the particulate material.

FIG. 3 is a partial view showing a wetting syringe connected to the vented syringe in the wetting or post-wetted configuration with the plunger seal retracted to the proximal side of the vent holes

FIG. 4 is a partial expanded view of an alternate embodiment of the plunger and seal assembly.

FIG. 5 is an expanded and partially exposed view of another alternate embodiment of the plunger and seal assembly.

### DETAILED DESCRIPTION OF THE INVENTION

As described herein, the invention comprises a multi-component system and method of use that, when used with a suitable fluid, permits and promotes the preparation and delivery of a particulate material for use as a bone graft device. A preferred embodiment of the invention is a multi-component syringe system, the system being supplied either empty or pre-filled with a desired bone graft material. The multi-component syringe device 10 is further defined as comprising a syringe barrel 12, a syringe plunger 11, and a nozzle member 13. The components preferably are pre-assembled and pre-filled with the desired bone graft particulate material 21 needing mixing with a wetting solution prior to use. The system further comprises a wetting syringe 30 or similar device for expelling liquid that is structured so as to temporarily mate or connect with the distal end of the vented syringe 10 such that liquid may be forced from the wetting syringe 30 into the vented syringe 10.

The syringe barrel 12 primarily is cylindrical in form, having a forward or distal end and rearward or proximal end. Use of a translucent or clear material is preferred to permit visualization of the bone graft material content and the progress of fluid installation. The syringe plunger 11 is inserted through the proximal end of the syringe barrel 12. During use, the plunger 11 is pressed forward toward the distal or forward end of the barrel 12 in order to expel the wetted particulate material 21. The syringe plunger 11 is not capable of drawing sufficient liquid into the barrel 12 because of the presence of at least one vent hole 14.

The syringe plunger 11 generally is a straight plunger shaft—typically tubular, cylindrical or cross-shaped in cross-section. It may be composed of either a single material with suitable sealing properties, or' may be a two-component unit, consisting of a plunger shaft 16 and an attached polymeric or rubber plunger seal 15. The plunger 11 is designed to form a fluid-tight seal within the syringe barrel 12.

The nozzle member 13 generally is comprised of a polyethylene or polypropylene material. The nozzle member 13 is removably connected to the distal end of the syringe barrel 12. This removable connection is preferably obtained via a friction fit, although other means of connection such as screw threads also are encompassed within the invention. A cap member 17 may be mounted onto the nozzle member 13 to retain the particulate material 21. The nozzle member 13 may also feature a means 31 for the attachment of the wetting syringe 30 by which a wetting fluid is administered to the enclosed particulate material 21 at time of use.

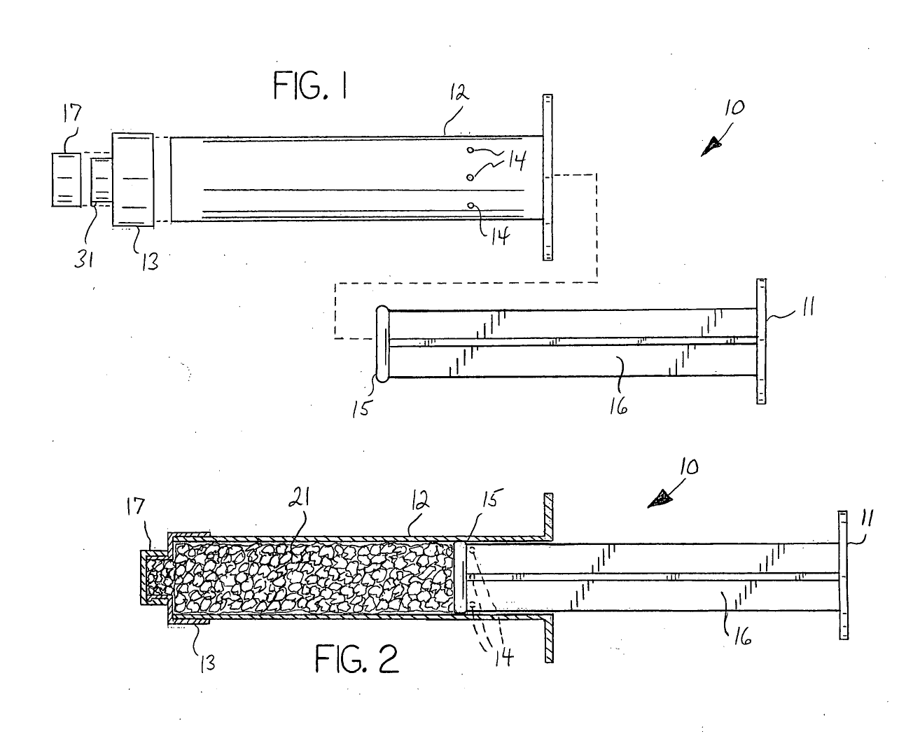

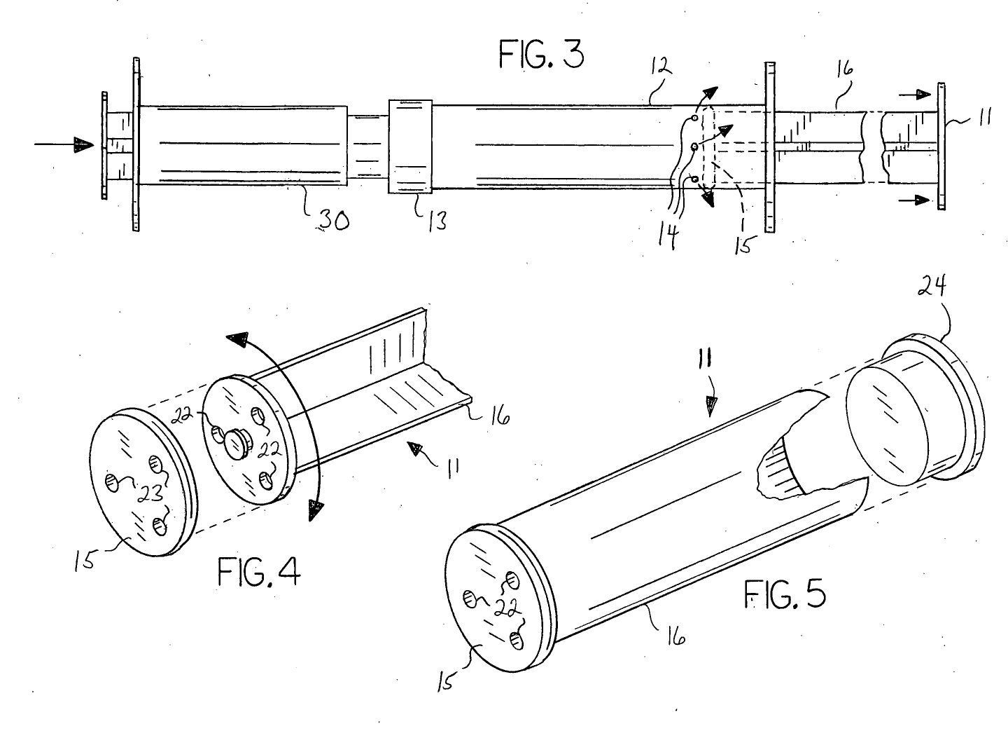

Although other formats are possible within the scope of this invention, one preferred embodiment of the device is described herewith and illustrated in FIGS. 1 through 3. The vented syringe 10 of this embodiment comprises a clear polypropylene barrel 12, with at least one and preferably a plurality of vent holes 14 spaced annularly near the proximal end of the barrel 12, a polyethylene plunger 11 with a rubber seal 15, and a polyethylene nozzle member 13. The nozzle member 13 is preferably friction fit onto the distal end of the barrel 12. As shown, the nozzle member 13 may also comprise an attachment means 31, such as a tabbed hub, which can engage a standard Luer-Lok® wetting syringe 30 during the wetting procedure. The device of this embodiment is preferably supplied fully assembled and pre-filled with a particulate bone graft material 21, such as Bioglass® osteostimulative bone graft particulate material. At time of supply or pre-wetting, the rubber seal 15 of the plunger 11 is positioned distally to the vent holes 14 so as to be between the bone graft particulate material 21 and the vent holes 14, and a cap member 17 is mounted onto the nozzle member 13 to retain the particulate material 21, as shown in FIG. 2. The bone graft material 21 is wetted by attaching a syringe or similar device 30 filled with a wetting fluid, such as blood, bone marrow, or saline, to the nozzle member 13 of the vented syringe 10. Upon expression of the fluid from the wetting syringe 30 through the nozzle member 13 and into the interior of the bone graft vented syringe 10, the increased pressure in the syringe 10 forces the plunger 11 in the proximal direction. Alternatively, the plunger 11 may be physically retracted a short distance in the barrel 12. In either manner, the plunger seal 15 is now positioned to the proximal side of the vent holes 14. The air resident within the particulate material 21 is now expelled through the vent holes 14 as the wetting solution is forced into the particulate material 21, as shown in FIG. 3. Once all air in the particulate material 21 has been replaced by the fluid and released through the vent holes 14, fluid will begin to flow from the open vent holes 14, at which time the fluid syringe 30 is removed and the syringe plunger 11 depressed to reseal the vent holes 14. To discharge or eject the wetted particulate material 21, the nozzle member 13 is removed from the barrel 12, if necessary, and the plunger 11 is advanced such that the wetted particulate material 21 is ejected from the distal end of the syringe 10.

In an alternate embodiment, the device comprises a polyethylene plunger 11 with a rubber seal 15, a clear polypropylene barrel 12, and a nozzle member 13. In this embodiment the barrel 12 is not vented through the wall. Instead, plunger vent holes 22 are placed in the end of the plunger shaft 16 and are closed with the rubber seal 15, which has a series of matching vent holes 23 that are rotatably offset from those in the plunger shaft 16, as shown in FIG. 4. Prior to attachment of the wetting syringe 30 to the nozzle member 13, the plunger shaft 16 is rotated to align the vent holes 22 in the shaft base with those in the plunger seal 15, permitting venting of gas from the syringe 10 when the fluid is administered. Once all air in the enclosed graft material 21 has been replaced by the fluid, the plunger shaft 16 is counter-rotated to reseal the vent holes 23. At time of bone graft material application, the nozzle member 13 is removed from the distal end of the barrel 12 and the wetted particulate material 21 expressed via the plunger.

In another alternative embodiment, the device consists of a plunger 11 and barrel 12 with a friction fit nozzle member 13. In this embodiment, the vent holes 22 are placed in the front or distal end of the plunger 11, the rear or proximal end being sealed with a rubber stopper 24, as shown in FIG. 5. At the time of attachment of the fluid syringe 30 to the nozzle member 13, the rubber stopper 24 is removed from the rear end of the plunger 11 to permit air venting during fluid administration. Once all air in the enclosed graft material 21 has been replaced by the fluid, the rubber plunger cap 24 is replaced to seal the system and the material 21 expressed.

As a functional overview, it is generally intended that the invention be supplied in a pre-assembled, pre-filled state. It is understood, however, that the device may be supplied empty, for filling in the surgical suite by any of a number of particulate bone graft devices available.

With respect to the above description, it is to be realized that the optimum relationship for the components of the invention, to include variations in component material, overall device size and volume, function and manner of mixing, operation, and use, are deemed readily apparent and obvious to one skilled in the art, and all equivalent relationships to those described in the specification are intended to be encompassed by the present invention.

Therefore, the foregoing is considered as illustrative only of the principles of the invention. Further, since modifications and changes will readily occur to those skilled in the art, it is not desired to limit the invention to the exact format and materials described, and accordingly, all suitable modifications and equivalents may be resorted to, falling within the scope of the claims of the invention.

We claim:

1. A wetted particulate containment and mixing vented syringe system comprising the combination of:
a vented syringe having a distal end and a proximal end, said vented syringe comprising a barrel, a plunger having a plunger seal disposed within said barrel, and at least one vent hole positioned on said barrel near said proximal end of said vented syringe, whereby said plunger is movable relative to said barrel such that said plunger seal resides either distally or proximally to said at least one vent hole;
particulate material disposed within said barrel of said vented syringe; and
a wetting syringe temporarily mountable to said distal end of said vented syringe, said wetting syringe capable of expelling a liquid into said vented syringe.

2. The system of claim 1, said vented syringe further comprising a nozzle member removably mounted onto said barrel at said distal end of said vented syringe, and further wherein said wetting syringe is temporarily mountable to said nozzle member.

3. The system of claim 2, further comprising a cap member removably mounted onto said nozzle member.

4. The system of claim 1, wherein said at least one vent hole comprises a plurality of vent holes annularly spaced on said barrel.

5. The system of claim 1, further comprising mechanical connecting means for temporarily connecting said wetting syringe and said vented syringe.

6. A system comprising the combination of:
a vented syringe having a distal end and a proximal end, said vented syringe comprising a barrel, a plunger having a plunger seal disposed within said barrel, and at least one vent hole positioned on said barrel near said proximal end of said vented syringe, whereby said plunger is movable relative to said barrel such that said plunger seal resides either distally or proximally to said at least one vent hole; and
a wetting syringe temporarily mountable to said distal end of said vented syringe, said wetting syringe capable of expelling a liquid into said vented syringe.

7. The system of claim 6, said vented syringe further comprising a nozzle member removably mounted onto said barrel at said distal end of said vented syringe, and further wherein said wetting syringe is temporarily mountable to said nozzle member.

8. The system of claim 7, further comprising a cap member removably mounted onto said nozzle member.

9. The system of claim 6, wherein said at least one vent hole comprises a plurality of vent holes annularly spaced on said barrel.

10. The system of claim 6, further comprising mechanical connecting means for temporarily connecting said wetting syringe and said vented syringe.

11. A method of mixing and ejecting a wetted particulate material comprising the steps of:
providing a vented syringe having a distal end and a proximal end, said vented syringe comprising a barrel, a plunger having a plunger seal disposed within said barrel, and at least one vent hole positioned on said barrel near said proximal end of said vented syringe, whereby said plunger is movable relative to said barrel such that said plunger seal resides either distally or proximally to said at least one vent hole;
inserting particulate material into said barrel of said vented syringe;
providing a wetting syringe containing a liquid;
temporarily connecting said wetting syringe to said distal end of said vented syringe;
expelling said liquid from said wetting syringe into said barrel of said vented syringe to produce wetted particulate material, whereby said plunger seal is moved toward said proximal end of said vented syringe such that said plunger seal is positioned proximally to said at least one vent hole such that gas within said barrel of said vented syringe is expelled through said at least one vent hole;
continuing to expel liquid from said wetting syringe into said barrel of said vented syringe until said liquid begins to pass through said at least one vent hole;
disconnecting said wetting syringe from said vented syringe; and
advancing said piston into said barrel of said vented syringe to eject said wetted particulate material from said vented syringe.

12. The method of claim 11, further comprising the steps of temporarily closing said distal end of said barrel in order to retain said particulate material within said barrel after said particulate material has been inserted into said barrel; and
and opening said distal end of said barrel prior to said step of temporarily connecting said wetting syringe to said distal end of said vented syringe.

13. The method of claim 11, further comprising the step of drawing liquid into said wetting syringe prior to said step of temporarily connecting said wetting syringe to said distal end of said vented syringe.

14. The method of claim 12, further comprising the step of drawing liquid into said wetting syringe prior to said step of temporarily connecting said wetting syringe to said distal end of said vented syringe.
